(19) United States
(12) Patent Application Publication
(10) Pub. No.: US 2015/0044671 A1
(43) Pub. Date: Feb. 12, 2015

(54) PROBES TARGETING THE GENE ENCODING THE SHIGA TOXIN AND USE THEREOF FOR DETECTION OF ENTEROHEMORRHAGIC ESCHERICHIA COLI (EHEC)

(71) Applicants: Grzegorz Wegrzyn, Gdansk (PL); Bozena Nejman-Falenczyk, Gdansk (PL)

(72) Inventors: Grzegorz Wegrzyn, Gdansk (PL); Bozena Nejman-Falenczyk, Gdansk (PL)

(73) Assignee: 3G THERAPEUTICS INC., Miami, FL (US)

(21) Appl. No.: 14/343,476

(22) PCT Filed: Sep. 7, 2012
(86) PCT No.: PCT/PL2012/000084

§ 371 Date: Mar. 7, 2014

(30) Foreign Application Priority Data
Sep. 9, 2011 (PL) P.396 283

(51) Int. Cl. C12Q 1/68

(52) U.S. Cl. CPC C12Q 1/689; C12Q 2600/158
USPC 435/6.11; 536/24.32

(57) ABSTRACT

The invention relates to a method for detection of enterohemorrhagic Escherichia coli (EHEC), a probe for/the detection of enterohemorrhagic Escherichia coli (EHEC), sequences for the fragment of the gene encoding the Shiga toxin, the use of probes and sequences. More particularly, the invention relates to a method of visual detection and diagnostics of enterohemorrhagic Escherichia coli (EHEC) by detecting the signal from a probe using a transilluminator that emits UV radiation. Invention also relates to a method of visual detection and diagnosis of any other pathogen and/or biological material by detecting the signal from a probe using a transilluminator that emits UV radiation.

The invention relates to a method for detection of enterohemorrhagic Escherichia coli (EHEC), a probe for the detection of enterohemorrhagic Escherichia coli (EHEC), sequences for the amplification of a fragment of the gene encoding the Shiga toxin, the use of probes and sequences. More particularly, the invention relates to a method of visual detection and diagnostics of enterohemorrhagic Escherichia coli (EHEC) by detecting the signal from a probe using a transilluminator that emits UV radiation. The method also relates to visual detection and diagnostics of pathogens and other biological, materials by detecting the signal from a probe using a transilluminator that emits UV radiation.

Due to the fact that patients infected with EHEC differ significantly from those infected by the majority of common bacterial strains, proper identification of the pathogen is crucial when Shiga toxin production is suspected, which requires rapid diagnostic procedures, which is not an easy task.

Although the EHEC phenotype has been correlated with the O157 serotype, and in fact the majority of infections by strains producing the Shiga toxin relates to this serotype, it is clear that E. coli serotypes other than the O157 may be responsible for this phenotype [1]. Therefore, methods of detecting the presence of the O157 serotype might be insufficient. Earlier studies have shown that EHEC does not cause fermentation of sorbitol [2, 3]; therefore, one of the recommended tests was the use of sorbitol McConkey plates for the detection of E. coli sorbitol-negative strains. Subsequent studies have indicated, however, that some strains of E. coli O157 are sorbitol-positive; and therefore, the conclusion is presented that it is not possible to determine an EHEC phenotype by microbiological or immunological measurements due to the lack of correlation between the serotype and the phenotype [1-3]. For this reason, only genetic testing may be appropriate for the unique identification of EHEC.

Although the stx genes can be detected in clinical material without isolation of bacteria, the U.S. Centers for Disease Control and Prevention indicated significant flaws of solely using assays that are not based on a culture. Firstly, it does not isolate the infectious organism in order to determine the serotype and perform specific diagnostics. Secondly, the lack of an isolated organism limits the ability of a physician to predict the potential acuteness of infection and the risk for severe disease following contact with the patient and limits the capacity of public health services to detect and control outbreaks and to monitor trends in the epidemiology.

For this reason, specific guidelines are described for the procedures for the detection of EHEC [1]: (i) samples should be cultured on selective and differentiating agar plates; (ii) these plates should be simultaneously measured by a test that detects the Shiga toxins or the genes encoding these toxins; (iii) these isolates should be submitted as soon as possible to the state or local public health laboratories in order to perform further molecular characteristics; (iv) detection of EHEC or Shiga toxin should be reported immediately to the treating physician, to the public health laboratory for confirmation and subsequent isolation and study of the organism, and to the appropriate public health services for investigation.

Foregoing recommendations can be virtually used exclusively for the use of appropriate methods. The problem was that, so far, only a small number of laboratory tests were available that allow one to detect EHEC, and many of these were both time consuming and expensive. This was the reason why a large number of new tests for the detection of EHEC were developed over the last few years.

The first class of tests are new PCR-based tests for the detection of EHEC. In many cases, multiple PCR procedures were used, and the most widely used target genes are stx1, stx2 (encoding the Shiga toxin) and eae (encoding intimin), while genes encoding different O antigens were also often included. Specific assays are different, but usually they take from one to several hours, and their sensitivity ranges from 10² to 10⁶ bacterial cells (in particular, colony forming units, CFU) per reaction, corresponding to 10⁴-10⁸ cfu/mL of the sample [4-18].

In several approaches, the authors used the Real Time PCR (RT-PCR) technique, which can give not only the qualitative result but also the quantitative result. This is a clear advantage of these methods, especially in combination with the use of ultra-fast RT-PCR procedures, which provide results in less than 10 minutes. In addition, the detection limit can be between 3 and 50 equivalents of the genome, which is important in view of the small number of EHEC cells capable of inducing infection in the intestine. The problem is, however, the relatively high cost of equipment for RT-PCR and the often complicated process of optimising the reaction conditions. This makes it difficult to implement these methods in ordinary hospital laboratories. However, some EHEC detection procedures, based on RT-PCR, appear to be potentially useful in both specific and very important analyses.

An interesting possibility is the use of the diagnostic technique LAMP (loop-mediated isothermal amplification). LAMP-based assays can even detect the presence of solitary pathogenic cells or viruses in one hour. Although this method is not very popular, it may provide a promising alternative. It is important that such a sensitive method for detection can be useful in detecting pathogens whose infectious dose is as low as 100 or less, as in the case of EHEC. Additionally, LAMP can be combined with other methods such as the RT-PCR method or FRET (Fluorescence Resonance Energy Transfer) [19-22].

Another class of tests is based on novel methods of nucleic acid hybridisation. These include the use of a fully automated, electric biochip, colorimetric detection method based on the photopolymerisation and DNA microarrays [23-25]. These novel hybridisation methods are sophisticated and effective; however, they may still be too difficult to implement in a standard clinical laboratory.

A new approach is the use of nanoparticles for detection of both EHEC and the Shiga toxin. Due to the fact that the Shiga toxin can specifically interact with the nanoparticle Gal-α1,4-Gal glycopolydiacetylene 1.4 (GPDA) or globotriose-functionalised gold nanoparticles (AuNP), it can be used to detect these proteinaceous proteins [26-27]. In addition, the use of carbon nanoparticles in “lateral flow” (immunoassay “lateral flow” for the nucleic acids) strips was proposed to detect genes encoding the Shiga toxin [28]. Gold nanoparticles were also used in the modified method for detecting the stx2 gene in the colorimetric assay [29]. Again, though sophisticated, these methods can be widely used in clinical laboratories in the future rather than at the present.

Improved immunoassays for detecting EHEC, Shiga toxin-converting phages or Shiga-like toxins are under development, in particular combined with other methods, such as PCR or the use of an electrical test biochip or extracellular translation tests [30-40]. Additionally, improved classical methods based on a bacterial culture are considered by some authors to be especially advantageous in the correct identification of EHEC, particularly along with molecular assays [41, 42].

Lastly, a very interesting method based on the efficient induction of Shiga toxin-converting prophages has been described recently [43]. Prophage detection was possible after filtration of the culture medium (using a 96-well plate to test a number of cultures at the same time) and norfloxacin treatment (to induce prophages) along with follow-up PCR reactions specific for the phage gene. This procedure may be useful in detecting EHEC and in further detailed studies of bacteriophages that have genes encoding the Shiga toxin. It can also be assumed that this method can be used in predicting the virulence of E. coli isolates.

Patent application EP 2 292 799 A1 (published Mar. 9, 2011) discloses a process for molecular risk assessment (MRA) in a sample which is suspected of having the presence of Escherichia coli encoding the Shiga toxin (STEC), including such steps as: contacting the said sample, or the isolated DNA, with a pair of primers derived from the replacing target genes stx1, stx2 and eae, wherein the process is characterised in that it also comprises contacting the sample, or the isolated DNA, with a pair of primers derived from the following target genes nleB, nleH1-2, nleE, ent/espL2 and detecting the presence or absence of an amplification product for each of these target genes.

In applications US20050282194A1 (published 22 Dec., 2005), EP1380655A2 (published 19 Oct., 1998) and US20030215814A1 (published 20 Nov., 2003), a method is disclosed for the detection of Shiga toxin-producing organisms or toxins of the Shiga toxin type, in particular E. coli producing Shiga toxin type toxins in biological samples using RT-PCR. The invention also presents the primers and probes for the detection of Shiga toxin-producing organisms or toxins of the Shiga toxin type, as well as a product containing these primers and probes for the detection of these organisms.

Whereas in the application WO9848046A2 (published 1998 Oct. 29) and patent US6664080B1 (published 16 Dec., 2003), there is presented a method for detecting pathogenic E. coli in a sample comprising PCR amplification of the DNA isolated from said sample using specific oligonucleotide primers.

In light of the above-mentioned information, there is still a need for a single EHEC detection method that would be characterised by simplicity, speed, sensitivity and low cost. Therefore, it is a choice between cheap and easy procedures of not very high, but still acceptable sensitivity (probably the most useful in ordinary hospital laboratories) and the sophisticated and complex methods that give very accurate results (probably the most useful in research laboratories and large medical centres).

The aim of the solution according to the invention was to develop a new method of rapid detection and diagnosis of enterohemorrhagic Escherichia coli (EHEC), based on the PCR reaction using the 5′ exonuclease activity of Taq polymerase and TaqMan probes and detecting the signal from the above probes using a transilluminator that emits UV radiation.

The implementation of such a solution for a particular purpose and resolution of the problems described in the prior art led to the development and delivery of a method characterised by a new mode of detection, which distinguishes it from the previously described methods that used complex and expensive equipment, such as Real-Time PCR machines and plate readers. The solution indicated in this disclosure allows for visual assessment of the presence of enterohemorrhagic bacteria E. coli (EHEC) carrying the Shiga toxin 1 and/or 2 and the type of the toxin carried by the bacteria using a standard UV transilluminator.

The invention relates to a method for detecting enterohemorrhagic Escherichia coil (EHEC), characterised in that it is based on the PCR reaction using the 5′ exonuclease activity of Taq polymerase and TaqMan probes and the detection of the signal from the probe using a transilluminator that emits UV radiation, wherein the probes are short oligonucleotides complementary to the genes of Shiga toxins 1 and 2, which contain at the 5′ end the fluorescent reporter FAM (6-carboxyfluorescein), and at the 3′ end the fluorescence quencher molecule BHQ-1.

Preferably, when in a) the first stage, the PCR reaction is carried out using TaqMan probes and the 5′ exonuclease activity of Taq polymerase (5′ nuclease assay), and during the PCR reaction, the probe binds to the complementary sequence on the template DNA and is degraded at the elongation stage by the Taq polymerase having a 5′ exonuclease activity, which is followed by separation of the two molecules and fluorescent light emission, and in b) the second stage, after the PCR reaction is completed, the test tube in which the reaction was performed is placed on the transilluminator, which is the source of ultraviolet radiation at or about 312 nm, and the UV light induces fluorescence of the fluorescein derivative FAM released from the effect of the BHQ-1 quencher, and a greenish-yellow luminescence of the solution in the test tube is observed.

Preferably, the probes are short oligonucleotides complementary to the genes of Shiga toxins 1 and 2, which contain at the 5′ end the fluorescent reporter FAM (6-carboxyfluorescein), and at the 3′ end the fluorescence quencher molecule BHQ-1 and are defined as Seq. ID NOs 3 and 6.

Preferably, the sequences of the gene encoding the Shiga toxin 1 are described by Seq. ID No. 1 and/or Seq. ID No. 2.

Preferably, the sequences of the gene encoding the Shiga toxin 2 are described by Seq. ID No. 4 and/or Seq. ID No. 5.

Preferably, the fluorescence is visible only when the labelled probe will be degraded, and thus when the DNA complementary to the designed probes is introduced to the test tube.

Another subject of the invention is a method for detecting pathogens and other biological materials, characterised in that the method is based on the PCR reaction using the 5′ exonuclease activity of Taq polymerase and TaqMan probes and the detection of the signal from the probe using a transilluminator that emits UV radiation, wherein the probes are short oligonucleotides complementary to the genes of pathogens and other biological materials, which contain at the 5′ end the fluorescent reporter FAM (6-carboxyfluorescein), and at the 3′ end the fluorescence quencher molecule BHQ-1.

Another subject of the invention is a probe for the detection of enterohemorrhagic Escherichia coli (EHEC), characterised in that it is a short oligonucleotide of no more than 80 bp, complementary to the genes of Shiga toxin 1 and/or 2, which contains at the 5′ end the fluorescent reporter FAM (6-carboxy-fluorescein) and at the 3′ end the fluorescence quencher molecule BHQ-1 (Black Hole Quencher).

Preferably, the probe is a sequence corresponding to a fragment of the gene encoding the Shiga toxin 1 or 2.

Preferably, the probe sequence is selected from Seq. ID No. 3 or Seq. ID No. 6.

Another subject of the invention is the sequence for the amplification of the fragment of the gene of Shiga toxin 1, characterised in that it is described by Seq. ID No. 1 or Seq. ID No. 2.

Another subject of the invention is the sequence for amplification of the fragment of a gene encoding the Shiga toxin 2, characterised in that it is described by Seq. ID No. 4 or Seq. ID No. 5.

Another subject of the invention is the use of the probes as defined above for the detection and/or diagnostics of enterohemorrhagic Escherichia coli (EHEC).

Another subject of the invention is the use of the sequences as defined above for the detection and/or diagnostics of enterohemorrhagic Escherichia coli (EHEC).

For a better understanding of the invention, the disclosure is partially shown in the figures, where:

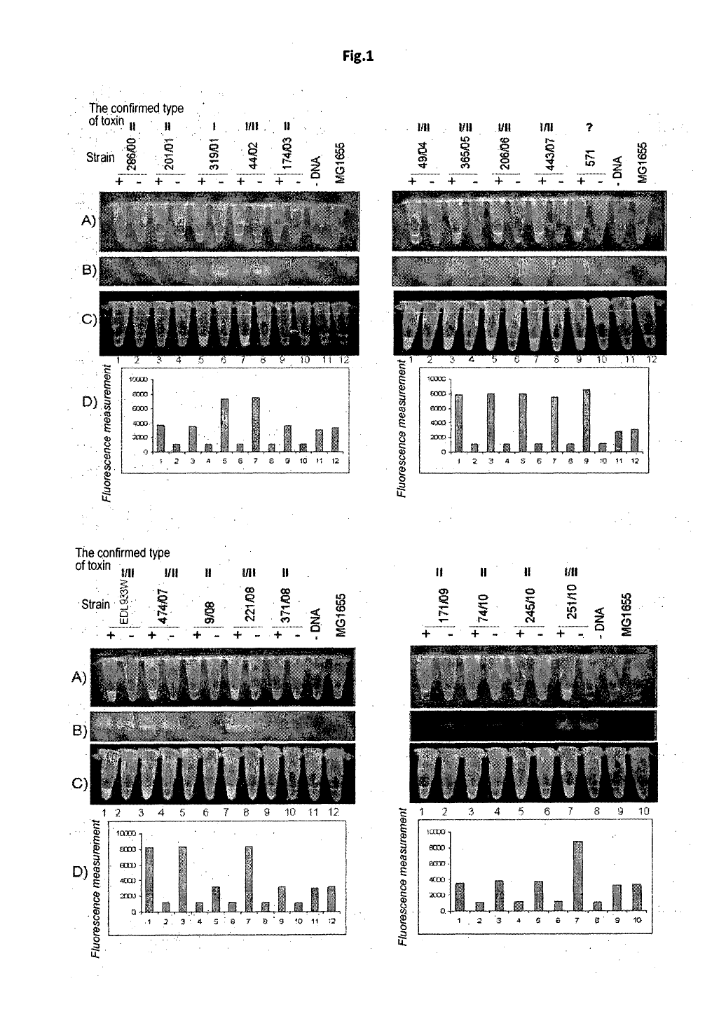

FIG. 1 shows the analysis of 19 EHEC strains for the presence of the gene encoding Shiga toxin 1 using a PCR reaction with a TaqMan probe (+). Detection of the signal from the probe: tox1probe, complementary to the gene encoding Shiga toxin 1 A) using the gel documentation system (Gel Doc XR—Bio-Rad) C) and UV transilluminator at a wavelength of 312 nm. B) Analysis of PCR products by agarose gel electrophoresis. D) Measurement of the fluorescence signal at 485/535 nm using a Perkin Elmer Multilabel Counter Victor3 reader, Wallac 1420. The control reaction performed without a probe (−), without the template DNA (−DNA), and the reaction performed with genomic DNA of the E. coli MG1655 strain not containing genes coding for Shiga toxin 1 and 2.

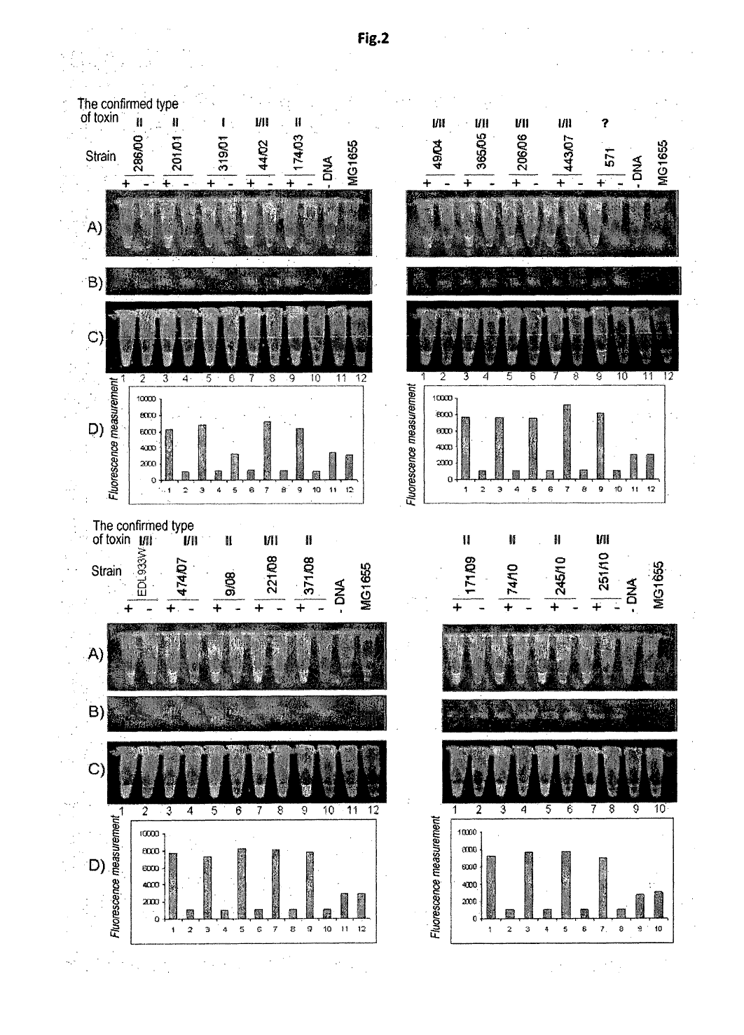

FIG. 2 shows the analysis of 19 EHEC strains for the presence of the gene encoding Shiga toxin 2 using a PCR reaction with a TaqMan probe (+). Detection of the signal from the probe: tox2probe, complementary to the gene encoding Shiga toxin 2 A) using the gel documentation system (Gel Doc XR—Bio-Rad) C) and UV transilluminator at a wavelength of 312 nm. B) Analysis of PCR products by agarose gel electrophoresis. D) Measurement of the fluorescence signal at 485/535 nm using a Perkin Elmer Multilabel Counter Victor3 reader, Wallac 1420. The control reaction performed without a probe (−), without the template DNA (−DNA), and the reaction performed with genomic DNA of the E. coli MG1655 strain not containing genes coding for Shiga toxin 1 and 2.

The embodiments of the invention are presented below. The method described in the invention can be also used for detection of any other pathogen or biological material.

### EMBODIMENTS OF THE INVENTION

The present method consists of the fact that in the first step, PCR reaction is carried out using TaqMan probes and the 5′ exonuclease activity of Taq polymerase (5′ nuclease assay). As used herein, the specific probes, defined by sequences described in SEQ. ID NO: 3 and 6, are short oligonucleotides complementary to the genes of Shiga toxins 1 and 2, which contain at the 5′ end the fluorescent reporter FAM (6-carboxyfluorescein), and at the 3′ end the fluorescence quencher molecule BHQ-1 (Black Hole Quencher). As the quencher and the fluorochrome are in close contact, there is no fluorescent signal emission. During the PCR reaction using a primer pair (ID No. 1-2 or No. 4-5) and probes (ID No. 3, respectively, and ID NO: 6), each probe binds to the complementary sequence on the template DNA (gene stx1 and stx2 respectively) and is degraded during the elongation stage by Taq polymerase having a 5′ exonuclease activity, which causes fluorochrome separation from the quencher. Separation of the two molecules allows for emission of fluorescent light. In the second stage, after completion of the PCR reaction, a 0.2 ml test tube in which the reaction was performed is placed on the transilluminator with the source of ultraviolet radiation at or about 312 nm. UV light induces the fluorescence of fluorescein derivative FAM, released from the effect of the BHQ-1 quencher, allowing for the observation, of a greenish-yellow solution luminosity in a test tube (perceptible by eye). The greenish-yellow fluorescence is visible only when the labelled probe will be degraded, and thus when the DNA complementary to the designed probes (ID No 3 and 6) and primers (ID No 1-2 and 4-5) is introduced to the test tube.

### Isolation of DNA by Boiling Lysis

5 ml of bacteria incubated o/n in LB at 37° C. was centrifuged at 10 000 rpm for 10 min. The pellet was resuspended in 250 μl of distilled water, boiled for 10 min at 100° C., and centrifuged once again at 10 000 rpm for 5 min. The supernatant was diluted 10× in distilled water and stored at −20° C. Aliquots of 2 μl of diluted DNA (100-300 ng) were used for PCR.

DNA Isolation by Lysis with Lysozyme and Proteinase K

The template DNA was isolated from 1 ml of fresh cultures in liquid tryptic soy broth (TSB). After 4 h incubation at 37° C. with aeration (300 rpm), the culture was centrifuged. The bacterial pellet was mixed with 40 μl of 10 mg/ml lysozyme and incubated for 30 min at 37° C. 160 μl of lysis buffer (10 mM Tris-HCl, pH 8.0, 10 mM EDTA and 1% Triton X-100) containing 2 mg/ml proteinase-K was added, and the tubes were placed at 52° C. for at least 1 h or until the suspension becomes transparent. Next, 0.8 ml of 10 mM Tris-HCl, pH 8.0, was added, and the tubes were heated at 95° C. for 30 min. This DNA was diluted 10× with distilled water, and two microliters were used in the PCR reaction.

### PCR Reactions

In experiments with the Taq polymerase DFS-Bioron 250 ng of the template DNA and MgCl2 at a concentration of 4 mM were used for the reaction, the reaction proceeded correctly. In experiments with SuperHotTaq DNA Polymerase Bioron signal detection was possible while using 130 ng of DNA (all other parameters, as well as the program, were unchanged. Reaction buffer “incomplete” is the trade name for the DFS-Taq polymerase (Bioron), the buffer does not contain magnesium hence the “incomplete” and that is why it is essential to add magnesium separately.

### Example 1

### Detection of the Bacterial Shiga Toxin 1 Gene, PCR Reaction Conditions

| | Final | |
|---|---|---|
| | concentration or | |
| Component | amount | |
| DNA | 250 | ng |
| 10x Reaction buffer “incomplete” (Bioron) | 1x | |
| dNTPs mix (4 mM of each dNTP) | 160 | μM (each) |
| MgCl₂ (Bioron) | 4 | mM |
| Primer F: tox1F | 1 | μM |
| Primer R: tox1R | 1 | μM |
| Probe: tox1probe (diluted approximately 20x) | 150 | nM |
| DFS-Taq DNA polymerase (Bioron) | 1.25 | U |
| H₂O | to 25 | μl |

Program

| | | | |
|---|---|---|---|
| 95° C. - 2 min | | | |
| 95° C. - 15 s | | | Product length tox1 = 196 bp |
| | {close oversize brace} | 28 cycles | |
| 60° C. - 60 s | | | |

When the reaction is complete, the tubes in which the reaction was performed (for example, the tubes Nippon of a 0.2 ml volume, strips of tubes with the flat lid) are removed from the thermocycler (Eppendorf), briefly centrifuged and placed in a transilluminator, where a UV light with a wavelength of 312 nm is used for induction (Vilber Lourmat). The characteristic greenish-yellow fluorescence indicates the presence of EHEC Shiga toxin gene 1.

### Example 2

### Detection of the Bacterial Shiga Toxin 2 Gene, PCR Reaction Conditions

| | Final | |
|---|---|---|
| | concentration or | |
| Component | amount | |
| DNA | 250 | ng |
| 10x Reaction buffer “incomplete” (Bioron) | 1x | |
| dNTPs mix (4 mM of each dNTP) | 160 | μM (each) |
| MgCl₂ (Bioron) | 4 | mM |
| Primer F: tox2F | 1 | μM |
| Primer R: tox2R | 1 | μM |
| Probe: tox2probe (diluted approximately 20x) | 150 | nM |
| DFS-Taq DNA polymerase (Bioron) | 1.25 | U |
| H₂O | to 25 | μl |

Program

| | | | |
|---|---|---|---|
| 95° C. - 2 min | | | |
| 95° C. - 15 s | | | Product length tox2 = 211 bp |
| | {close oversize brace} | 28 cycles | |
| 60° C. - 60 s | | | |

When the reaction is complete, the tubes in which the reaction was performed (for example, the tubes Nippon of a 0.2 ml volume, strips of tubes with the flat lid) are removed from the thermocycler (Eppendorf), briefly centrifuged and placed in a transilluminator, where a UV light with a wavelength of 312 nm is used for induction (Vilber Lourmat). The characteristic greenish-yellow fluorescence indicates the presence of EHEC Shiga toxin gene 2.

### Examples of Use of the Method

The developed method was tested on 19 samples. 17 were obtained from the Państwowy Zakfad Higieny (PHZ). DNA samples were isolated from the verotoxigenic rods of E. coli strains isolated from the faeces of patients in Poland. The presence of verotoxin was determined by using the PZH cytotoxicity assay on a Vero cell line, the RPLA-VTEC test (Oxoid) and standard PCR assays [18, 32]. The table below presents a list of samples obtained from the PZH. Two additional DNA samples (571 and EDL933W) came from the own collection. The results were further confirmed by checking the presence of the product by agarose gel electrophoresis and by measuring the fluorescence of 485 nm/535 nm using a plate reader (Perkin Elmer Multilabel Counter Victor3). The DNA from the samples obtained from PZH were isolated as described above using lysis with lysozyme and proteinase-K, and other samples were isolated by boiling lysis (10 min. 100° C.).

TABLE 1
List of DNA samples obtained from the PZH.

| Strain No | Serotype | Shiga toxin 1 | Shiga toxin 2 |
|---|---|---|---|
| 286/00 | O157 | 0 | 1 |
| 44/02 | O157 | 1 | 1 |
| 174/03 | O157 | 0 | 1 |
| 49/04 | O157 | 1 | 1 |
| 365/05 | O157 | 1 | 1 |
| 206/06 | O157 | 1 | 1 |
| 443//07 | O157 | 1 | 1 |
| 474/07 | O157 | 1 | 1 |
| 9/08 | O157 | 0 | 1 |
| 221/08 | O157 | 1 | 1 |
| 371/08 | O157 | 0 | 1 |
| 171/09 | O157 | 0 | 1 |
| 74/10 | O157 | 0 | 1 |
| 245/10 | O111 | 0 | 1 |
| 251/10 | O157 | 1 | 1 |
| 201/01 | O26 | 0 | 1 |
| 319/01 | O26 | 1 | 0 |

### REFERENCES

1. Gould L H, Bopp C, Strockbine N et al.: Recommendations for diagnosis of Shiga toxin-producing Escherichia coli infections by clinical laboratories. MMWR Recomm. Rep. 58(RR-12), 1-14 (2009).
2. Riley L W, Remis R S, Helgerson S D, McGee H B, Wells J G, Davis B R: Hemorrhagic colitis associated with a rare Escherichia coli serotype. N. Engl. J. Med. 308(12), 681-685 (1983).
3. Blanco J E, Blanco M, Alonso M P et al.: Serotypes, virulence genes of Shiga toxin-producing Escherichia coli isolates from human patients. J. Clin. Microbiol. 42(1), 311-319 (2004).
4. Persson S, Olsen K E, Scheutz F, Krogfelt K A, Gerner-Smidt P: A method for fast and simple detection of major diarrhoeagenic Escherichia coli in the routine diagnostic laboratory. Clin. Microbiol. Infect. 13(5), 516-524 (2007).
5. Riyaz-Ul-Hassan S, Syed S, Johri S, Verma V, Qazi G N: Application of a multiplex PCR assay for the detection of Shigella, Escherichia coli and Shiga toxin-producing E. coli in milk. J. Dairy Res. 76(2), 188-194 (2009).
6. Mull B, Hill V R: Recovery and detection of Escherichia coli O157:H7 in surface water, using ultrafiltration and real-time PCR. Appl. Environ. Microbiol. 75(11), 3593-3597 (2009).
7. Fratamico P M, DebRoy C, Miyamoto T, Liu Y: PCR detection of enterohemorrhagic Escherichia coli O145 in food by targeting genes in the E. coli O145 O-antigen gene cluster and the Shiga toxin 1 and Shiga toxin 2 genes. Foodborne Pathog. Dis. 6(5), 605-611 (2009).
8. Rajkhowa S, Das R, Bora S, Rajkhowa C, Rahman H, Bujarbaruah K M: Detection of Shiga toxin-producing Escherichia coli and enteropathogenic Escherichia coli in faecal samples of healthy mithun (Bos frontalis) by multiplex polymerase chain reaction. Zoonoses Public Health. 57(6), 397-401 (2010).
9. Gómez-Duarte O G, Arzuza O, Urbina D et al.: Detection of Escherichia coli enteropathogens by multiplex polymerase chain reaction from children's diarrheal stools in two Caribbean-Colombian cities. Foodborne Pathog. Dis. 7(2), 199-206 (2010).
10. Rooks D J, Yan Y, McDonald J E, Woodward M J, McCarthy A J, Allison H E: Development and validation of a qPCR-based method for quantifying Shiga toxin-encoding and other lambdoid bacteriophages. Environ. Microbiol. 12(5), 1194-1204 (2010).
11. Chui L, Couturier M R, Chiu T et al.: Comparison of Shiga toxin-producing Escherichia coli detection methods using clinical stool samples. J. Mol. Diagn. 12(4), 469-475 (2010).
12. Cunningham S A, Sloan L M, Nyre L M, Vetter E A, Mandrekar J, Patel R: Three-hour molecular detection of Campylobacter, Salmonella, Yersinia, and Shigella species in faeces with accuracy as high as that of culture. J. Clin. Microbiol. 48(8), 2929-2933 (2010).
13. DebRoy C, Roberts E, Davis M, Bumbaugh A: Multiplex polymerase chain reaction assay for detection of nonserotypable Shiga toxin-producing Escherichia coli strains of serogroup O147. Foodborne Pathog. Dis. 7(11), 1407-1414 (2010).
14. Bonetta S, Borelli E, Bonetta S, Conio O, Palumbo F, Carraro E: Development of a PCR protocol for the detection of Escherichia coli O157:H7 and Salmonella spp. in surface water. Environ. Monit. Assess. 177(1-4), 493-503 (2011).
15. Park S H, Hanning I, Jarquin R et al.: Multiplex PCR assay for the detection and quantification of Campylobacter spp., Escherichia coli O157:H7, and Salmonella serotypes in water samples. FEMS Microbiol. Lett. 316(1), 7-15 (2011).
16. Bernini V, Sgarbi E, Bove C G, Gatti M, Neviani E: A polyphasic approach to detect enterotoxigenic Staphylococcus aureus and diarrheagenic Escherichia coli in raw milk Italian cheeses by multiplex PCR. J. Food Prot. 73(12), 2281-2284 (2010).
17. Bugarel M, Beutin L, Scheutz F, Loukiadis E, Fach P: Identification of genetic markers for differentiation of Shiga toxin-producing, enteropathogenic and avirulent strains of Escherichia coli O26. Appl. Environ. Microbiol. 77(7), 2275-2281 (2011).
18. Chassagne L, Pradel N, Robin F, Livrelli V, Bonnet R, Delmas J: Detection of stx1, stx2, and eae genes of enterohemorrhagic Escherichia coli using SYBR Green in a real-time polymerase chain reaction. Diagn. Microbiol. Infect. Dis. 64(1), 98-101 (2009).
19. Hara-Kudo Y, Nemoto J, Ohtsuka K et al.: Sensitive and rapid detection of Vero toxin-producing Escherichia coli using loop-mediated isothermal amplification. J. Med. Microbiol. 56(3), 398-406 (2007).
20. Ohtsuka K, Tanaka M, Ohtsuka T, Takatori K, Hara-Kudo Y: Comparison of detection methods for Escherichia coli O157 in beef livers and carcasses. Foodborne Pathog. Dis. 7(12), 1563-1567 (2010).
21. Kouguchi Y, Fujiwara T, Teramoto M, Kuramoto M: Homogenous, real-time duplex loop-mediated isothermal amplification using a single fluorophore-labeled primer and an intercalator dye: Its application to the simultaneous detection of Shiga toxin genes 1 and 2 in Shiga toxigenic Escherichia coli isolates. Mol. Cell. Probes. 24(4), 190-195 (2010).
22. Hara-Kudo Y, Konishi N, Ohtsuka K et al.: Detection of Verotoxigenic Escherichia coli O157 and O26 in food by plating methods and LAMP method: a collaborative study. Int. J. Food Microbiol. 122(1-2), 156-161 (2008).
23. Los M, Los J M, Wegrzyn G: Rapid identification of Shiga toxin-producing Escherichia coli (STEC) using electric biochips. Diagn. Mol. Pathol. 17(3), 179-184 (2008).
24. Schuurman T, Roovers A, van der Zwaluw W K et al.: Evaluation of 5′-nuclease and hybridization probe assays for the detection of Shiga toxin-producing Escherichia coli in human stools. J. Microbiol. Methods. 70(3), 406-415 (2007).
25. Quinones B, Swimley M S, Taylor A W, Dawson E D: Identification of Escherichia coli O157 by using a novel colorimetric detection method with DNA microarrays. Foodborne Pathog. Dis. 8(6), 705-711 (2011).
26. Nagy J O, Zhang Y, Yi W et al.: Glycopolydiacetylene nanoparticles as a chromatic biosensor to detect Shiga-like toxin producing Escherichia coli O157:H7. Bioorg. Med. Chem. Lett. 18(2), 700-703 (2008).
27. Chien Y Y, Jan M D, Adak A K et al.: Globotriose-functionalized gold nanoparticles as multivalent probes for Shiga-like toxin. Chembiochem. 9(7), 1100-1109 (2008).
28. Noguera P, Posthuma-Trumpie G A, van Tuil M et al.: Carbon nanoparticles in lateral flow methods to detect genes encoding virulence factors of Shiga toxin-producing Escherichia coli. Anal. Bioanal. Chem. 399(2), 831-838 (2011).
29. Jyoti A, Pandey P, Singh S P, Jain S K, Shanker R: Colorimetric detection of nucleic acid signature of Shiga toxin producing Escherichia coli using gold nanoparticles. J. Nanosci. Nanotechnol. 10(7), 4154-4158 (2010).
30. Los M, Los J M, Blohm L et al.: Rapid detection of viruses using electrical biochips and anti-virion sera. Lett. Appl. Microbiol. 40(6), 479-485 (2005).
31. Hajra T K, Bag P K, Das S C, Mukherjee S, Khan A, Ramamurthy T: Development of a simple latex agglutination assay for detection of Shiga toxin-producing Escherichia coli (STEC) by using polyclonal antibody against STEC. Clin. Vaccine Immunol. 14(5), 600-604 (2007).
32. Stefan A, Scaramagli S, Bergami R et al.: Real-time PCR and enzyme-linked fluorescent assay methods for detecting Shiga-toxin-producing Escherichia coli in mincemeat samples. Can. J. Microbiol. 53(3), 337-342 (2007).
33. Nielsen K, Smith P, McRae H, Yu W, Widdison J: Detection of Escherichia coli O157:H7 by fluorescence polarization assay and polymerase chain reaction. J. Immunoassay Immunochem. 28(3), 251-265 (2007).
34. Teel L D, Daly J A, Jerris R C et al.: Rapid detection of Shiga toxin-producing Escherichia coli by optical immunoassay. J. Clin. Microbiol. 45(10), 3377-3380 (2007).
35. Zhang W, Bielaszewska M, Pulz M et al.: New immuno-PCR assay for detection of low concentrations of Shiga toxin 2 and its variants. J. Clin. Microbiol. 46(4), 1292-1297 (2008).
36. Willford J, Mills K, Goodridge L D: Evaluation of three commercially available enzyme-linked immunosorbent assay kits for detection of Shiga toxin. J. Food Prot. 72(4), 741-747 (2009).
37. He X, Quinones B, Carter J M, Mandrell R E: Validation of a cell-free translation assay for detecting Shiga toxin 2 in bacterial culture. J. Agric. Food Chem. 57(11), 5084-5088 (2009).
38. Rozand C, Feng P C: Specificity analysis of a novel phage-derived ligand in an enzyme-linked fluorescent assay for the detection of Escherichia coli O157:H7. J. Food Prot. 72(5), 1078-1081 (2009).
39. Verstraete K, De Zutter L, Messens W, Herman L, Heyndrickx M, De Reu K: Effect of the enrichment time and immunomagnetic separation on the detection of Shiga toxin-producing Escherichia coli O26, O103, O111, O145 and sorbitol positive O157 from artificially inoculated cattle faeces. Vet. Microbiol. 145(1-2), 106-112 (2010).
40. Hermos C R, Janineh M, Han L L, McAdam A J: Shiga toxin-producing Escherichia coli in children: detection and clinical manifestations of O157:H7 and non-O157:H7 infection. J. Clin. Microbiol. 49(3), 955-959 (2011).
41. Hu J, Green D, Swoveland J, Grant M, Boyle D S: Preliminary evaluation of a procedure for improved detection of Shiga toxin-producing Escherichia coli in fecal specimens. Diagn. Microbiol. Infect. Dis. 65(1), 21-26 (2009).
42. Couturier M R, Lee B, Zelyas N, Chui L: Shiga-toxigenic Escherichia coli detection in stool samples screened for viral gastroenteritis in Alberta, Canada. J. Clin. Microbiol. 49(2), 574-578 (2011).
43. McDonald J E, Smith D L, Fogg P C, McCarthy A J, Allison H E: High-throughput method for rapid induction of prophages from lysogens and its application in the study of Shiga toxin-encoding Escherichia coli strains. Appl. Environ. Microbiol. 76(7), 2360-2365 (2010).
44. Development of a rapid, high-throughput method for induction of Shiga toxin-converting prophages has been described. This method may be potentially useful in both basic and epidemiological studies on these phages, as well as in the course of developing novel diagnostic procedures of EHEC infections.

1. A method for detecting enterohemorrhagic Escherichia coli (EHEC), characterised in that it is based on the PCR reaction using the 5′ exonuclease activity of Taq polymerase and TaqMan probes and the detection of the signal from the probe using a transilluminator that emits UV radiation, wherein the probes are short oligonucleotides complementary to the genes of Shiga toxins 1 and 2, which contain at the 5′ end the fluorescent reporter FAM (6-carboxyfluorescein), and at the 3′ end the fluorescence quencher molecule BHQ-1.

2. A method according to claim 1, characterised in that in a) the first stage, the PCR reaction is carried out using TaqMan probes and the 5′ exonuclease activity of Taq polymerase (5′ nuclease assay), and during the PCR reaction, the probe binds to the complementary sequence on the template DNA and is degraded at the elongation stage by the Taq polymerase having a 5′ exonuclease activity, which is followed by separation of the two molecules and fluorescent light emission, and in b) the second stage, after the PCR reaction is completed, the test tube in which the reaction was performed is placed on the transilluminator, which is the source of ultraviolet radiation at or about 312 nm, and the UV light induces fluorescence of the fluorescein derivative FAM released from the effect of the BHQ-1 quencher, and a greenish-yellow luminescence of the solution in the test tube is observed.

3. A method according to claim 1, characterised in that in the probes are short oligonucleotides complementary to the genes of Shiga toxins 1 and 2, which contain at the 5′ end the fluorescent reporter FAM (6-carboxyfluorescein), and at the 3′ end the fluorescence quencher molecule BHQ-1 and are defined as Seq. ID Nos. 3 and 6.

4. A method according to claim 1, characterised in that sequences for the gene encoding Shiga toxin 1 are described by Seq. ID No. 1 and/or Seq. ID No. 2.

5. A method according to claim 1, characterised in that sequences for the gene encoding Shiga toxin 2 are described by Seq. ID No. 4 and/or Seq. ID No. 5.

6. A method according to claim 1, characterised in that the fluorescence is visible only when the labelled probe will be degraded, and thus when the DNA complementary to the designed probes is introduced to the test tube.

7. A method for detecting pathogens and other biological materials, characterised in that the method is based on the PCR reaction using the 5′ exonuclease activity of Taq polymerase and TaqMan probes and the detection of the signal from the probe using a transilluminator that emits UV radiation, wherein the probes are short oligonucleotides complementary to the genes of pathogens and other biological materials, which contain at the 5′ end the fluorescent reporter FAM (6-carboxyfluorescein), and at the 3′ end the fluorescence quencher molecule BHQ-1.

8. A probe for the detection of enterohemorrhagic Escherichia coli (EHEC), characterised in that it is a short oligonucleotide of no more than 80 bp, complementary to the genes of Shiga toxin 1 and/or 2, which contains at the 5′ end the fluorescent reporter FAM (6-carboxy-fluorescein), and at the 3′ end the fluorescence quencher molecule BHQ-1 (Black Hole Quencher).

9. A probe according to claim No. 7, characterised in that the probe is a sequence for detection of the gene encoding the Shiga toxin 1 or 2.

10. A probe according to claim No. 7, characterised in that the probe is chosen from sequences Seq. ID No. 3 or Seq. ID No. 6.

11. The sequence for the amplification of the gene encoding Shiga toxin 1, characterised in that it is described by Seq. ID No. 1 or Seq. ID No. 2.

12. The sequence for the amplification of the gene encoding Shiga toxin 2, characterised in that it is described by Seq. ID No. 4 or Seq. ID No. 5.

13. A method of using the probe according to claim 8 comprising: using the probe to detect or carry out diagnostics of enterohemorrhagic Escherichia coli (EHEC).

14. A method of using the probe according to claim 9 comprising: using the probe to detect or carry out diagnostics of enterohemorrhagic Escherichia coli (EHEC).

15. A method of using the probe according to claim 10 comprising: using the probe to detect or carry out diagnostics of enterohemorrhagic Escherichia coli (EHEC).


SEQUENCE LISTING (6 sequences)

SEQ ID NO: 1 - DNA, 27 bp, Escherichia coli
  source 1..27: /mol_type=“DNA” /organism=“Escherichia coli”
gacgatacctttacagttaaagtgggt

SEQ ID NO: 2 - DNA, 21 bp, Escherichia coli
  source 1..21: /mol_type=“DNA” /organism=“Escherichia coli”
tctccgcctgctattttcact

SEQ ID NO: 3 - DNA, 32 bp, Escherichia coli
  source 1..32: /mol_type=“DNA” /organism=“Escherichia coli”
aatcttcagtctcttcttctcagtgcgcaaat

SEQ ID NO: 4 - DNA, 25 bp, Escherichia coli
  source 1..25: /mol_type=“DNA” /organism=“Escherichia coli”
ttccaagtataatgaggatgacaca

SEQ ID NO: 5 - DNA, 21 bp, Escherichia coli
  source 1..21: /mol_type=“DNA” /organism=“Escherichia coli”
cccacataccacgaatcaggt

SEQ ID NO: 6 - DNA, 29 bp, Escherichia coli
  source 1..29: /mol_type=“DNA” /organism=“Escherichia coli”
aatctgcaaccgttactgcaaagtgctca